(19) United States
(12) Patent Application Publication
(10) Pub. No.: US 2022/0008007 A1
(43) Pub. Date: Jan. 13, 2022

(54) MICRONEEDLE ARRAY AND SENSOR INCLUDING THE SAME

(71) Applicant: ICREATE TECHNOLOGY (ZHUHAI) CO., LTD., Zhuhai (CN)

(72) Inventors: SZE KEE TAM, Hong Kong (CN); YUE YUE ZHAO, Hong Kong (CN); KA MING NG, Hong Kong (CN); KA YIP FUNG, Hong Kong (CN); YONG YU, Hong Kong (CN); CHEUK HEI HERRY MAK, Hong Kong (CN); CE YAN, Hong Kong (CN); LINGDA XU, Hong Kong (CN)

(73) Assignee: ICREATE TECHNOLOGY (ZHUHAI) CO., LTD., Zhuhai (CN)

(21) Appl. No.: 17/373,137

(22) Filed: Jul. 12, 2021

Related U.S. Application Data
(60) Provisional application No. 63/102,951, filed on Jul. 13, 2020.

(51) Int. Cl. A61B 5/00; A61B 5/145; A61B 5/15

(52) U.S. Cl. CPC A61B 5/685; A61B 2562/028; A61B 5/150984; A61B 5/14514

(57) ABSTRACT

The microneedle array includes a substrate having a central opening formed therethrough, and a plurality of microneedles positioned about a perimeter defining the central opening. At least one of the microneedles has a recess formed therein adjacent a tip thereof, and this recess is at least partially filled with a layer of active material. A sensor for detecting chemical analytes, biological analytes or the like may be constructed by providing two such microneedle arrays, with one serving as the working electrode and one serving as a reference electrode. The working electrode and the reference electrode may both be connected to a signal analyzer for detecting electrochemical signals. The working electrode and the reference electrode may be separate from one another or may be stacked together.

### CROSS-REFERENCE TO RELATED APPLICATION

This application claims the benefit of U.S. Provisional Patent Application No. 63/102,951, filed on Jul. 13, 2020.

### BACKGROUND

### 1. Field

The disclosure of the present patent application relates to the detection and sensing of biological and/or chemical analytes, and particularly to a microneedle array used as an electrode in biochemical sensors.

### 2. Description of the Related Art

Microneedle devices are commonly used for extracting and/or detecting biological fluids, such as glucose, lactate, cholesterol, creatinine, etc., in a minimally-invasive, painless and convenient manner. Microneedle devices allow biological fluids to be sensed or withdrawn from the body (i.e., in vivo), particularly from, or through, skin or other tissue barriers with minimal or no damage, pain or irritation to the tissue.

Microneedles have been integrated into biosensors for detecting particular biomarkers. Typically, a micron-sized electrochemical biosensor probe is inserted within a cavity formed in a hollow microneedle. Such devices, however, are typically costly and difficult to manufacture, particularly due to the great difficulties involved in the manufacture of nano-scale sensors, which often involves nano-scale deposition techniques to be performed on silicon wafers and the like.

It would obviously be desirable to be able to manufacture a sensor for the same purposes, but without the difficulty involved in first manufacturing a nano-scale sensor and then embedding that nano-scale sensor within a microneedle. Thus, a microneedle array and a sensor including the same solving the aforementioned problems are desired.

### SUMMARY

The microneedle array may be used as an electrode for sensing, for example, biological or chemical analytes in a biological fluid. The microneedle array includes a substrate having a central opening formed therethrough, and a plurality of microneedles positioned about a perimeter defining the central opening. At least one of the microneedles has a recess formed therein adjacent a tip thereof, and this recess is at least partially filled with a layer of active material. The substrate may be substantially planar, with each of the microneedles projecting substantially perpendicular to the plane of the substrate. The plurality of microneedles may be aligned such that they all project in the same direction.

The substrate and each of the microneedles may be formed from a metal or a biocompatible polymer, and may further be coated with a dielectric layer. Non-limiting examples of such metals include titanium, stainless steel, gold and platinum. The active material is dependent upon the particular analyte to be detected. Non-limiting examples of such active materials include biomarker recognition materials, anti-interference materials, immobilized enzymes, electrochemical reference materials, and combinations thereof.

In order to make the microneedle array, the base material of the substrate is first cut and trimmed to define the outer contour of the substrate and the overall size of the microneedle array. The central opening is then formed through the substrate. The central opening is formed irregularly, such that the plurality of microneedles are formed from the substrate and defined by the formation of the central opening. At this stage, the plurality of microneedles are positioned about the perimeter defining the central opening, with the plurality of microneedles lying within the plane of the substrate and projecting inwardly toward a center of the central opening.

The substrate and the plurality of microneedles are then coated with the dielectric material, and the recess is formed in at least one of the microneedles, adjacent a tip thereof. The recess is at least partially filled with the layer of active material, and the plurality of microneedles are bent such that they project perpendicular to the plane of the substrate. The plurality of microneedles may be bent such that they all project in the same direction.

Additionally, a sensor for detecting chemical analytes, biological analytes or the like may be constructed by providing two such microneedle arrays, with one serving as the working electrode and one serving as a reference electrode. The working electrode is constructed as in the previous embodiment, including a first substrate having a first central opening formed therethrough, and a plurality of first microneedles positioned about a perimeter defining the first central opening. At least one of the first microneedles has a first recess formed therein adjacent a tip thereof. A layer of a first active material at least partially fills the first recess. Similarly, the reference electrode includes a second substrate having a second central opening formed therethrough, with a plurality of second microneedles positioned about a perimeter defining the second central opening. At least one of the second microneedles has a second recess formed therein adjacent a tip thereof, with a layer of a second active material at least partially filling the second recess.

The working electrode and the reference electrode may then both be connected to a signal analyzer, or any other suitable device for detecting electrochemical signals, such as a voltmeter or the like. The working electrode and the reference electrode may be separate from one another or may be stacked together, such that the plurality of second microneedles projects through the first central opening, or vice versa.

These and other features of the present subject matter will become readily apparent upon further review of the following specification.

### BRIEF DESCRIPTION OF THE DRAWINGS

FIG. 1 is a perspective view of a microneedle array.

FIG. 2 is an elevational view of a single microneedle of the microneedle array.

FIG. 3 is a cross-sectional view of the microneedle of FIG. 2, taken along cross-sectional cut lines 3-3.

FIG. 4A, FIG. 4B, FIG. 4C, FIG. 4D and FIG. 4E illustrate successive steps of a process for manufacturing the microneedle array.

FIG. 5A is a perspective view of a sensor including two microneedle arrays.

FIG. 5B is a perspective view of an alternative embodiment of the sensor of FIG. 5A.

Similar reference characters denote corresponding features consistently throughout the attached drawings.

### DETAILED DESCRIPTION OF THE PREFERRED EMBODIMENTS

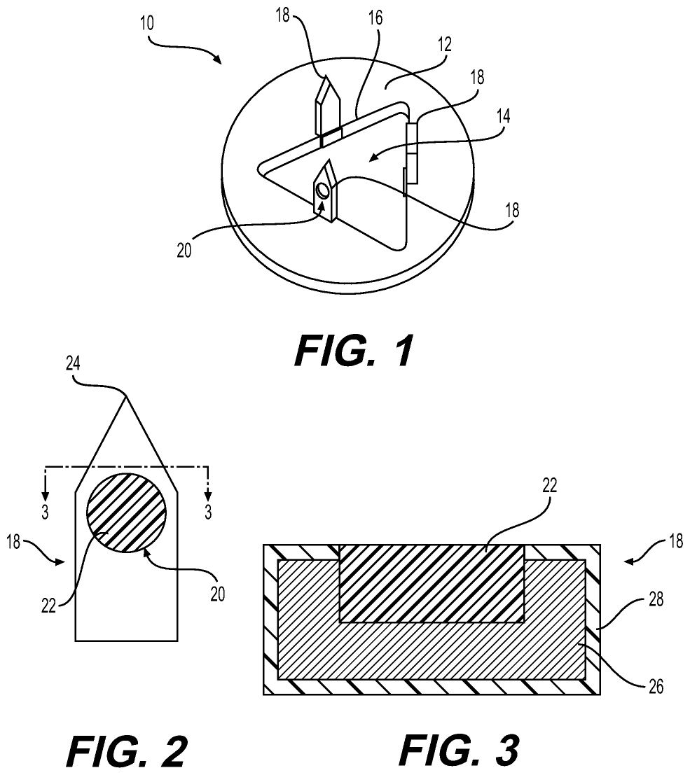

The microneedle array 10 may be used as an electrode for sensing, for example, biological or chemical analytes in a biological fluid. As shown in FIG. 1, the microneedle array 10 includes a substrate 12 having a central opening 14 formed therethrough, and a plurality of microneedles 18 positioned about a perimeter 16 defining the central opening 14. It should be understood that the circular shape of substrate 12 is shown for exemplary purposes only, and that substrate 12 may have any suitable shape and relative dimensions. Similarly, it should be understood that the substantially triangular central opening 14 is shown for exemplary purposes only, and that central opening 14 may have any suitable shape and relative dimensions. For the non-limiting example of FIG. 1, each side of the triangular central opening 14 may have a length of approximately 550 μm, although it should be understood that this dimension is provided as a non-limiting example only.

At least one of the microneedles 18 has a recess 20 formed therein adjacent a tip 24 thereof, and this recess 20 is at least partially filled with a layer of active material 22. Referring to FIGS. 2 and 3, it should be understood that the substantially rectangular shape (with a triangular tip 24) of each microneedle 18 is shown for exemplary purposes only, and that each microneedle 18 may have any suitable shape and relative dimensions. As noted above, at least one of microneedles 18 has a recess 20 formed therein. It should be understood that any number of the microneedles 18 may have recesses 20 formed therein, up to and including all of the microneedles 18. In FIGS. 2 and 3, the recess 20 is substantially circular, although it should be understood that the circular shape of recess 20 is shown for exemplary purposes only, and that recess 20 may have any suitable shape and relative dimensions. Non-limiting examples of alternative shapes include rectangles, hexagons and the like. Corresponding to the above non-limiting example for central opening 14, the maximum width of each microneedle 18 (measured in the horizontal direction in the orientation of FIG. 2) may be approximately 200 μm, the height of each microneedle 18 (measured in the vertical direction in the orientation of FIG. 2) may be approximately 480 μm, the angle of tip 24 may be approximately 55°, and the diameter of recess 20 may be approximately 150 μm. It should be understood that these dimensions are non-limiting examples only. Alternatively, the height of each microneedle 18 may, for example, range between 10 μm and 1000 μm, such as between 100 μm and 500 μm. As a further non-limiting example, the diameter of recess 20 may range between approximately 1 μm and 500 μm. For a rectangular recess, the former diameter may represent the longer side of the recess, and a shorter side of the recess may have a length between approximately 1 μm and 200 μm.

The substrate 12 may be substantially planar, as shown, with each of the microneedles 18 projecting substantially perpendicular to the plane of the substrate 12. The plurality of microneedles 18 may be aligned such that they all project in the same direction; i.e., they each project on the same side of substrate 12. It should be understood that substrate 12 may have any overall contour, and is not limited to a purely planar configuration. Further, it should be understood that microneedles 18 may, alternatively, project at angles therefrom and are not required to be purely perpendicular to the substrate 12.

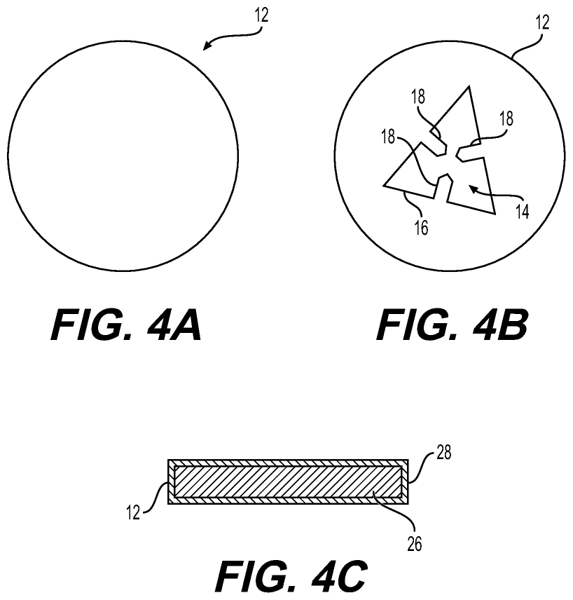

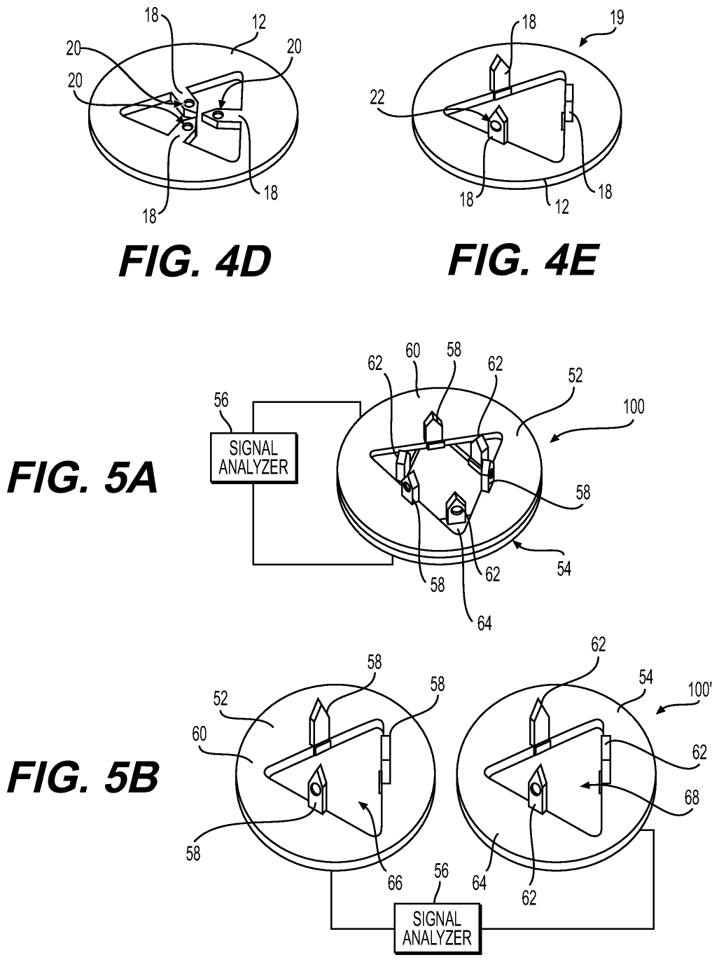

The substrate 12 and each of the microneedles 18 may be formed from a metal or a biocompatible polymer. Corresponding to the non-limiting exemplary dimensions given above, the metal or biocompatible polymer of substrate 12 and microneedles 18 may have a non-limiting exemplary thickness of approximately 100 μm. As shown in FIGS. 3 and 4C, the metal or biocompatible polymer of substrate 12 and microneedles 18 may further be coated with a dielectric layer 28. Corresponding to the non-limiting exemplary dimensions given above, the dielectric layer 28 may have a non-limiting exemplary thickness of approximately 25 μm. Additionally, corresponding to the non-limiting exemplary dimensions given above, the recess 20 may have a non-limiting exemplary depth of approximately 80 μm within the metal or biocompatible polymer (measured in the vertical direction in the orientation of FIG. 3), with an additional depth of 25 μm through the dielectric layer 28. Alternatively, recess 20 may have an exemplary depth of approximately 20% to approximately 90% the thickness of the microneedle 18.

It should be understood that substrate 12 and microneedles 18 may be made from any suitable type of electroconductive and biocompatible metal, biocompatible polymer, and/or at least one biocompatible polymer coated or plated with at least one electroconductive and biocompatible metal. Non-limiting examples of such metals include titanium, stainless steel, gold and platinum. The choice of the active material 22 which at least partially fills recess 20 is dependent upon the particular analyte to be detected. Non-limiting examples of such active materials 22 include biomarker recognition materials, anti-interference materials, immobilized enzymes, electrochemical reference materials, and combinations thereof. Non-limiting examples of anti-interference materials include semi-permeable materials, such as Nafion™ (C₇HF₁₃O₅S·C₂F₄) and/or polyurethane. Electrochemical reference materials may include one or more chemical layers to function as a redox electrode to maintain the redox potential of the electrode.

In order to make the microneedle array 10, the base material of the substrate 12 is first cut and trimmed to define the outer contour of the substrate 12 and the overall size of the microneedle array 10, as shown in FIG. 4A. The central opening 14 is then formed through the substrate 12. As shown in FIG. 4B, the central opening 14 is formed irregularly, such that the plurality of microneedles 18 are formed from the substrate 12 and defined by the formation of the central opening 14. At this stage, as shown in FIG. 4B, the plurality of microneedles 18 are positioned about the perimeter 16, which defines the central opening 14, with the plurality of microneedles 18 lying within the plane of the substrate 12 and projecting inwardly toward a center of the central opening 14.

As shown in FIG. 4C, the substrate 12 and the plurality of microneedles 18 are then coated with the dielectric material 28 and, as shown in FIG. 4D, the recess 20 is formed in at least one of the microneedles 18, adjacent the tip thereof. As shown in FIG. 3, the recess is at least partially filled with the layer of active material 22 and, as shown in FIG. 4E, the plurality of microneedles 18 are bent such that they project perpendicular to the plane of the substrate 12. The plurality of microneedles 18 may be bent such that they all project in the same direction, as discussed above.

Additionally, as shown in FIGS. 5A and 5B, a sensor 100, 100′ for detecting chemical analytes, biological analytes or the like may be constructed by providing two such microneedle arrays, with one serving as the working electrode 52 and one serving as a reference electrode 54. The working electrode 52 is constructed as in the previous embodiment, including a first substrate 60 having a first central opening 66 formed therethrough, and a plurality of first microneedles 58 positioned about a perimeter defining the first central opening 66. As in the previous embodiment, at least one of the first microneedles 58 has a first recess formed therein adjacent a tip thereof. A layer of a first active material at least partially fills the first recess. Similarly, the reference electrode 54 includes a second substrate 64 having a second central opening 68 formed therethrough, with a plurality of second microneedles 62 positioned about a perimeter defining the second central opening 68. At least one of the second microneedles 62 has a second recess formed therein adjacent a tip thereof, with a layer of a second active material at least partially filling the second recess.

The working electrode 52 and the reference electrode 54 may then both be connected to a signal analyzer 56, or any other suitable device for detecting electrochemical signals, such as a voltmeter or the like. In sensor 100 of FIG. 5A, the working electrode 52 and the reference electrode 54 are stacked together, such that the plurality of second microneedles 62 projects through the first central opening 66, or vice versa. Alternatively, in sensor 100′ of FIG. 5B, the working electrode 52 and the reference electrode 54 remain separated from one another. As is conventionally know, by measuring changes in potential, for example, using signal analyzer 56, the analyte, biomarker, etc. may be detected. It should be understood that the measured signal may be processed using any conventional techniques, such as, but not limited to, digitizing the signal and transforming the raw data of the signal into an indicator of biomarker concentration.

### EXAMPLE 1

For a sensor for detection of glucose, grade 1 pure titanium sheeting with a thickness of 100 μm was used to form the substrate and microneedles. The titatnium sheeting was cut into a circular shape with a diameter of 8 mm. A substantially triangular central opening, with each side having a length of 550 μm, was cut, leaving three microneedles (one for each side), each with a maximum width of 100 μm, similar to that shown in FIG. 4B. The titanium surfaces were then cleaned and coated with a parylene dielectric coating using chemical vapor deposition (CVD). The dielectric coating had a thickness of approximately 25 μm.

A circular recess was cut into each microneedle using laser engraving. The circular recess had a depth of 105 μm (penetrating through the 25 μm parylene coating layer and 80 μm into the titanium), with a diameter of 150 μm. The circular recess was laser-engraved at the middle of each microneedle. For the laser engraving (i.e., laser ablation), the laser intensity was in the range of 88.5% to 96.5% (with the preferred value being approximately 94.5%); the repetition rate was in the range of 40-50 kHz (with the preferred value being approximately 45 kHz); and the scan speed was in the range of 300-500 mm/s (with the preferred value being approximately 450 mm/s).

For the working electrode, the active layer was applied to fill the recess using inkjet printing of glucose oxidase (or glucose dehydrogenase), polyurethane and Nafion^{TM} ink. A separate reference electrode was prepared in an identical manner to that described above, but for the reference electrode, the active layer was applied to fill the recess using inkjet printing of Ag/AgCl ink. For each electrode, the microneedles were then bent to project perpendicular to the corresponding substrate, as in FIG. 4E.

### EXAMPLE 2

For a sensor for detection of lactate, a pure gold sheet with a thickness of 100 μm was used to form the substrate and microneedles. The gold sheet was cut into a circular shape with a diameter of 8 mm. A substantially triangular central opening, with each side having a length of 550 μm, was cut, leaving three microneedles (one for each side), each with a maximum width of 100 μm, similar to that shown in FIG. 4B. The gold surfaces were then cleaned and coated with a parylene dielectric coating using chemical vapor deposition (CVD). The dielectric coating had a thickness of approximately 25 μm.

A circular recess was cut into each microneedle using laser engraving. The circular recess had a depth of 105 μm (penetrating through the 25 μm parylene coating layer and 80 μm into the titanium), with a diameter of 150 μm. The circular recess was laser-engraved at the middle of each microneedle. For the laser engraving (i.e., laser ablation), the laser intensity was in the range of 88.5% to 96.5% (with the preferred value being approximately 94.5%); the repetition rate was in the range of 40-50 kHz (with the preferred value being approximately 45 kHz); and the scan speed was in the range of 350-500 mm/s (with the preferred value being approximately 470 mm/s).

For the working electrode, the active layer was applied to fill the recess using inkjet printing of lactate oxidase, polyurethane and Nafion™ ink. A separate reference electrode was prepared in an identical manner to that described above, but for the reference electrode, the active layer was applied to fill the recess using inkjet printing of Ag/AgCl ink. For each electrode, the microneedles were then bent to project perpendicular to the corresponding substrate, as in FIG. 4E.

It is to be understood that the microneedle array and the sensor including the same are not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A microneedle array, comprising:
a substrate having a central opening formed therethrough;
a plurality of microneedles positioned about a perimeter defining the central opening, wherein at least one of the microneedles has a recess formed therein adjacent a tip thereof; and
a layer of active material filling the recess.

2. The microneedle array as recited in claim 1, wherein the substrate is planar.

3. The microneedle array as recited in claim 2, wherein each of the microneedles projects perpendicular to the substrate.

4. The microneedle array as recited in claim 1, wherein the substrate and each of the microneedles is coated with a dielectric layer.

5. The microneedle array as recited in claim 1, wherein the substrate and the plurality of microneedles comprise a metal.

6. The microneedle array as recited in claim 5, wherein the metal is selected from the group consisting of titanium, stainless steel, gold and platinum.

7. The microneedle array as recited in claim 1, wherein the substrate and the plurality of microneedles comprise a biocompatible polymer.

8. The microneedle array as recited in claim 1, wherein the active material is selected from the group consisting of a biomarker recognition material, an anti-interference material, immobilized enzymes, an electrochemical reference material, and combinations thereof.

9. A method of making a microneedle array, comprising the steps of:
forming a central opening through a substrate, wherein a plurality of microneedles are positioned about a perimeter defining the central opening, the plurality of microneedles lying within a plane of the substrate and projecting inwardly toward a center of the central opening;
coating the substrate and the plurality of microneedles with a dielectric material;
forming a recess in at least one of the microneedles, adjacent a tip thereof;
filling the recess with a layer of active material; and
bending the plurality of microneedles to project perpendicular to the plane of the substrate.

10. A sensor, comprising:
a working electrode comprising:
a first substrate having a first central opening formed therethrough;
a plurality of first microneedles positioned about a perimeter defining the first central opening, wherein at least one of the first microneedles has a first recess formed therein adjacent a tip thereof; and
a layer of a first active material at least partially filling the first recess; and
a reference electrode comprising:
a second substrate having a second central opening formed therethrough;
a plurality of second microneedles positioned about a perimeter defining the second central opening, wherein at least one of the second microneedles has a second recess formed therein adjacent a tip thereof; and
a layer of a second active material at least partially filling the second recess.

11. The sensor as recited in claim 10, wherein each of the first and second substrates is planar.

12. The sensor as recited in claim 11, wherein each of the first microneedles projects perpendicular to the first substrate, and each of the second microneedles projects perpendicular to the second substrate.

13. The sensor as recited in claim 10, wherein the first substrate and each of the first microneedles is coated with a first dielectric layer.

14. The sensor as recited in claim 13, wherein the second substrate and each of the second microneedles is coated with a second dielectric layer.

15. The sensor as recited in claim 10, wherein the first substrate and the first plurality of microneedles comprise a first metal, and the second substrate and the second plurality of microneedles comprise a second metal.

16. The sensor as recited in claim 15, wherein each of the first metal and the second metal is selected from the group consisting of titanium, stainless steel, gold and platinum.

17. The sensor as recited in claim 10, wherein the first substrate and the first plurality of microneedles comprise a first biocompatible polymer.

18. The sensor as recited in claim 17, wherein the second substrate and the second plurality of microneedles comprise a second biocompatible polymer.

19. The sensor as recited in claim 10, wherein each of the first active material and the second active material is selected from the group consisting of a biomarker recognition material, an anti-interference material, immobilized enzymes, an electrochemical reference material, and combinations thereof.

20. The sensor as recited in claim 10, wherein the working electrode and the reference electrode are stacked, such that the plurality of second microneedles projects through the first central opening.
